(19) United States
(12) Patent Application Publication
(10) Pub. No.: US 2002/0016585 A1
(43) Pub. Date: Feb. 7, 2002

(54) Catheter with a bio-resorbable coating for preventing ascending infections

(76) Inventor: Hans E. Sachse, Nuernberg (DE)

Correspondence Address: Herbert B. Keil, KEIL & WEINKAUF, 1101 Connecticut Ave., N.W., Washington, DC, 20036, US

(21) Appl. No.: 09/916,294

(22) Filed: Jul. 30, 2001

(30) Foreign Application Priority Data
Jul. 29, 2000 (DE) P 100 37 353.4

(51) Int. Cl. A61M 27/00

(52) U.S. Cl. 604/544

(57) ABSTRACT

A catheter for removing urine from the bladder, or also for removal of other bodily fluids, which is of such type that ascending infections are prevented to the greatest degree possible. The catheter is designed in such a way that, when it is used for removing urine, the urethra is rinsed by the urine of the individual. A coating of the catheter walls with a substance which itself slowly dissolves in an aqueous environment assures that the surface of the catheter wall is continuously renewed and thereby prevents the spreading of germs on the surface of the catheter wall in the direction of the bladder.

### BACKGROUND OF THE INVENTION

Urethral catheters, which have a coating of antibiotically acting substances or which make use of the oligodynamic action of silver ions, for checking ascending infections are known. However, it has been found that the effects of such substances are very much limited in time. Furthermore, urethral catheters are known, by means of which it is intended to prevent ascending infections through a rinsing effect by means of the male's own urine. The urethral catheters in accordance with German patent applications P 44 27 443 and P 44 27 421 and U.S. Pat. No. 6,080,142 pursue this aim. In the course of scientific tests it has been shown, however, that bacteria migrate upward toward the bladder not only in the stagnating moist layer between the urethral wall and the outer wall of the catheter, but that this also occurs in the interior lumen of the catheter by growth along and on the catheter wall. The germs settle on the catheter wall and slowly spread in the direction of the body.

This is different in healthy urethral walls. Here, too, a continuous infection takes place from the direction of the always infected urethral orifice. But epithelial cells continuously flake off the surface of the healthy urethral wall. If germs ascending from the urethral orifice get on these epithelial cells of the urethral wall, they are rinsed out by the flow of urine when these epithelial cells flake off. They cannot remain in the urethra.

### SUMMARY OF THE INVENTION

The invention relates to a catheter for removing urine from the bladder, or also for removal of other bodily fluids, which is of such type that ascending infections are prevented to the greatest degree possible. The catheter is designed in such a way that, when it is used for removing urine, the urethra is rinsed by the urine of the individual. A coating of the catheter walls with a substance which itself slowly dissolves in an aqueous environment assures that the surface of the catheter wall is continuously renewed and thereby prevents the spreading of germs on the surface of the catheter wall in the direction of the bladder.

### BRIEF DESCRIPTION OF THE DRAWINGS

FIG. 1 is a longitudinal section through a catheter section located in the urethra.

### DETAILED DESCRIPTION OF THE INVENTION

A goal of the present invention is to design a catheter wall for preventing an ascending infection in such a way that the germs settling on the catheter wall cannot adhere to it and are rinsed out by the urine flow in the individual with the aid of a special rinsing catheter.

The catheter walls are coated with a bioresorbable coating (“sheathing”) for this purpose, which slowly dissolves in an aqueous environment, mainly by hydrolysis. Preferably, the catheter is made of an elastic tube with or without holding devices, with inlet openings in the area of its body for urine and other bodily fluids, being of such type that it can rinse the wall of a conduit in the body. Also preferably, the coating dissolves over a period of several weeks when the catheter is in use. As this process takes place on the surface of the coated catheter wall, the surface of the catheter wall is continuously renewed. In this way germs ascending the urethra on the catheter wall cannot maintain themselves on the catheter surface and are rinsed out by means of the rinsing mechanism of these special catheters.

In a preferred embodiment, the sheathing layer contains metallic silver particles which supply silver ions when they are released into the aqueous environment in the course of the dissolution process of the coated surface of the catheter wall. These silver ions provide a bactericidal effect and in this way additionally counteract an ascending infection.

In another preferred embodiment, in place of or in addition to the silver particles, there are antibiotics in the sheathing layer to increase the protective effects.

It may also be desirable for the sheathing to extend only over individual sections of the catheter, preferably over the sections with a rinsing effect.

In another embodiment, the entire catheter is made of a bioresorbable material, which is mainly dissolved by hydrolysis.

In yet another embodiment, the bioresorbable material is applied to a plastic mesh on the inside for increasing the stability.

Although the catheter has been primarily designed for the urethra, it can also be employed in other areas of the body which have an aqueous environment.

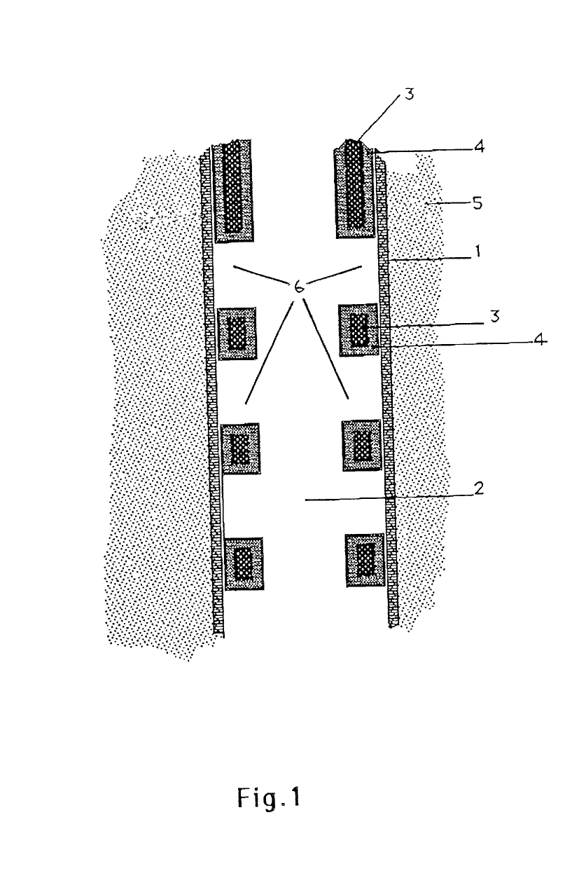

With reference to FIG. 1, the catheter shaft 3 has windows 6, through which the urine flowing in the catheter lumen 2 rinses the inner wall 1 of the urethra. The material of the catheter shaft 3 has been coated with a bioresorbable material 4. The wall of the urethra is identified by 5.

1. A catheter comprising an elastic tube with or without holding devices, with inlet openings in the area of its body for urine and other bodily fluids, being of such type that it can rinse the wall of a conduit in the body, characterized in that all the walls have been provided with a bioresorbable sheathing, which dissolves over a period of several weeks.

2. The catheter of claim 1, characterized in that metallic silver particles are embedded in the sheathing.

3. The catheter of claim 1, characterized in that portions of antibiotics are embedded in the sheathing.

4. The catheter of claim 1, characterized in that the entire catheter consists of a bioresorbable material, which is mainly dissolved by hydrolysis.

5. The catheter of claim 1, characterized in that the sheathing covers the walls of the catheter only partially.

6. The catheter of claim 5, characterized in that the sheathing covers the walls of the catheter only in the rinsing area.

7. The catheter of claim 1, wherein the catheter consists of a bioresorbable material, which has been applied to a plastic mesh for increased stability.

8. A process of catheterizing a patient, comprising inserting the catheter of claim 1 into a urethra of the patient and dissolving the bioresorbable sheathing over a period of several weeks.
